(19) United States
(12) Patent Application Publication
(10) Pub. No.: US 2021/0007969 A1
(43) Pub. Date: Jan. 14, 2021

(54) FAST-ABSORBING COSMETIC OIL THROUGH ACACIA OR OTHER TREE GUM DIFFUSION PROCESS

(71) Applicant: Lauren Servin, Maple Glen, PA (US)

(72) Inventor: Lauren Servin, Maple Glen, PA (US)

(21) Appl. No.: 16/961,083

(22) PCT Filed: Jan. 9, 2019
(86) PCT No.: PCT/IB19/50172

§ 371 Date: Sep. 23, 2020

Related U.S. Application Data
(60) Provisional application No. 62/615,073, filed on Jan. 9, 2018.

(51) Int. Cl. A61K 8/9789; A61K 8/92; A61K 8/06; A61Q 19/00; A61K 8/73

(52) U.S. Cl. CPC A61K 8/9789; A61K 8/92; A61K 8/737; A61Q 19/007; A61K 8/06

(57) ABSTRACT

An innovative fast-absorbing cosmetic oil is created through an acacia gum or other tree gum diffusion process. The lipophilic molecules from the polysaccharide chain of the tree gum chemically bond to the lipid molecules of the cosmetic oil. This diffusion process results in a composition of the fast-absorbing cosmetic oil that is substantially soft and velvety. The fast-absorbing cosmetic oil thus creates immediate plumping of the skin, fast absorption of oil into the skin without a greasy residue, skin tightening and lifting, and many other benefits to skin. Additionally, methods of making the composition of the fast-absorbing cosmetic oil are provided with or without the use of water and a broken emulsion process.

### FIELD OF THE INVENTION

The present invention relates generally to a cosmetic oil. More particularly, the present invention relates to a fast-absorbing cosmetic oil through an acacia gum or other tree gum diffusion process and methods of making the fast-absorbing cosmetic oil.

### BACKGROUND OF THE INVENTION

The outmost layers of human skin are of-based to act as a protective barrier in order to keep the skin safe from germs and dehydration. The human skin is considered the shield of a person's body and may become uncomfortable, disordered, or even damaged. When this shield is attacked or neglected, the human skin becomes unable to function properly to provide protection. It can become dull and dehydrated, dry and unevenly textured, open to developing forms of dermatitis and eczema, and sensitive, reactive, and reddened.

Cosmetic oils can help keep a user's skin healthy and attractive because they replenish the skin's natural oil content. Oils are among the most important cosmetic ingredients and are frequently used for a variety of external applications such as skin and hair care products. Oils not only form a temporary barrier, but they also feed skin with the nutrients it needs to restore itself to health. Specifically, the cosmetic oils are a natural source of vitamins and antioxidants and are high in skin-identical ingredients. These oils can offer a concentrated dose of omega fatty acids, that help repair skin's barrier function.

Despite all their benefits, cosmetic oils can be of a less desirable viscosity, color, texture, or even leave an oily residue on the user's skin or hair. Cosmetic oils can often be greasy and take time to absorb. Additionally, cosmetic oils are volatile and thus they begin to dissipate as soon as they are applied. It is critical to have a cosmetic oil that absorbs fast into the user's skin to minimize the greasy feeling and maximize the utilization and benefit of the oil. With the addition of acacia tree gum, the oiliness disappears, and absorption is significantly improved. However, acacia tree gum or other tree gums are normally emulsified in oil, but not diffused, to blend oil and water into an emulsified cream or lotion for making cosmetic products. Numerous innovations for cosmetic oils have been provided in the prior art. Even though these innovations may be suitable for the specific individual purposes to which they address, nevertheless, they may not be able to solve the problems of the user feeling greasy and the oils having low absorption rates.

Thus, an object of the present invention is to solve the aforementioned problems by providing a fast-absorbing cosmetic oil through an acacia gum or other tree gum diffusion process and methods of making thereof. The fast-absorbing cosmetic oil of the present invention incorporates the acacia gum or other tree gum into an existing cosmetic oil. Then the acacia gum or other tree gum diffuses into the cosmetic oil so as to form the fast-absorbing cosmetic oil through the acacia gum or other tree gum diffusion process. The acacia gum (or Gum Arabic) is a slightly acidic complex compound, made up of glycoprotein and polysaccharides and their calcium, magnesium and potassium salts. The principal polysaccharide is Arabic acid, a polysaccharide linking a D-galactose with branches composed of L-arabinose, L-rhamnose and D-glucuronic acids. Essentially, the proteins are classified as arabinogalactanes, rich in hydroxproline. Generally, acacia gum functions as a stabilizer in cosmetic products, where it changes the viscosity, imparts spreading properties and provides a protective coating and a smooth feel to the user. Through the diffusion process, the acacia gum used in the present invention is chemically reacted with the cosmetic oil to offer a fast-absorbing cosmetic oil that creates immediate plumping of the skin, fast absorption of oil into the skin without a greasy residue, and skin tightening and lifting. Further, the fast-absorbing cosmetic oil of the present invention acts as a natural barrier once applied to the user's skin or hair for improved moisture retention, and better supports the skin natural collagen and fibers.

### SUMMARY OF THE INVENTION

A fast-absorbing cosmetic oil is created through a diffusion process of a tree gum such as acacia gum or other tree gum into an existing cosmetic oil. The lipophilic molecules from the polysaccharide chain of the tree gum chemically bond to the lipid molecules of the cosmetic oil and thus diffuse across the cell membranes. This diffusion process results in a composition of the fast-absorbing cosmetic oil that is substantially soft and velvety. The fast-absorbing cosmetic oil thus creates immediate plumping of the skin, fast absorption of oil into the skin without a greasy residue, and skin tightening and lifting. Further, the fast-absorbing cosmetic oil acts as a natural barrier once applied to the user's skin or hair for improved moisture retention by preventing water loss, penetrates deeply to effectively reduce fine lines, makes the skin significantly supple with immediate plumping characteristics, builds skin cells over time, improves the overall structure of skin, and better supports the skin's natural collagen and fibers to promote a soft, healthy, and supple skin. Further, methods of making the composition of the fast-absorbing cosmetic oil are provided with or without the use of water and a broken emulsion process.

### BRIEF DESCRIPTION OF THE DRAWINGS

FIG. 1 is a flowchart illustrating the overall process followed by the preferred method of the present invention.

FIG. 2 is a flowchart illustrating the overall process followed by an alternative method of the present invention.

FIG. 3 is a flowchart illustrating the preferred sub-process for preparing a broken emulsion of the present invention.

FIG. 4 is a flowchart illustrating an alternative sub-process for preparing a broken emulsion of the present invention.

### DETAIL DESCRIPTIONS OF THE INVENTION

All illustrations of the drawings are for the purpose of describing selected versions of the present invention and are not intended to limit the scope of the present invention.

The present invention is a fast-absorbing cosmetic oil and a method of making the fast-absorbing cosmetic oil. Specifically, the present invention incorporates the acacia gum or other tree gum into an existing cosmetic oil. The acacia gum or other tree gum diffuses into the cosmetic oil to form the fast-absorbing cosmetic oil. The acacia gum (or Gum Arabic) is a slightly acidic complex compound, made up of glycoprotein and polysaccharides and their calcium, magnesium and potassium salts. The principal polysaccharide is Arabic acid, a polysaccharide linking a D-galactose with branches composed of L-arabinose, L-rhamnose and D-glucuronic acids. Essentially, the proteins are classified as arabinogalactanes, rich in hydroxproline. More specifically, the acacia gum is a natural product complex mixture of hydrophilic carbohydrate and hydrophobic protein components, and an emulsifier which absorbs into a cosmetic oil. Generally, acacia gum functions as a stabilizer in cosmetic products, where it changes the viscosity, imparts spreading properties and provides a protective coating for immune defense again ultraviolet (UV) radiation, and a smooth feel to the user.

Many existing cosmetic oils comprise a lot of Polyunsaturated acids such as Omega 3 (alpha linolenic acid) and Omega 6 fatty acids (linoleic acid and gamma-linoleic acid). When acacia tree gum or any other suitable tree gum is added to an existing cosmetic oil, the diffusion process starts: the negatively charged lipophilic molecules (tail) from the polysaccharide chain of the acacia tree gum chemically bond to the positively charged lipid molecules (head) of the oil. This chemical reaction continues until all lipid molecules of the oil are bonded to the lipophilic molecules of the acacia tree gum. The lipophilic molecules in a polysaccharide of the acacia tree gum are oil lipid soluble and thus diffuse across the cell membranes. This diffusion process results in a composition of the fast-absorbing cosmetic oil of the present invention, which is substantially soft and velvety. Through the diffusion process, the acacia gum used in the present invention is chemically reacted with the cosmetic oil to offer a fast-absorbing cosmetic oil that creates immediate plumping of the skin, fast absorption of oil into the skin without a greasy residue, and skin tightening and lifting. Further, the fast-absorbing cosmetic oil of the present invention acts as a natural barrier once applied to the user's skin or hair for improved moisture retention by preventing water loss. Additionally, the present invention penetrates deeply to effectively reduce fine lines, makes the skin significantly supple with immediate plumping characteristics, builds skin cells over time, improves the overall structure of skin, and better supports the skin natural collagen and fibers to promote a soft, healthy, and supple skin.

The preferred embodiment of the present invention comprises the acacia tree gum and an existing cosmetic oil. However, any suitable tree gum including, but not limited to, gum Arabic from the sap of Acacia trees, gum ghatti from the sap of Anogeissus trees, gum tragacanth from the sap of Astragalus shrubs, Karaya gum from the sap of Sterculia trees, etc., may be used in other embodiments of the present invention. Additionally, the existing cosmetic oil can be any suitable cosmetic oil including, but not limited to, Almond oil, Aloe Vera oil, Argan oil, Baobab oil, Chia seed oil, Coconut oil (liquid Flax seed oil, Grape seed oil, Jojoba oil, Manila oil, Meadowfoam oil, Moringa oil, Prickly Pear oil, Pomegranate Oil, Rosehip oil, etc. The composition of the fast-absorbing cosmetic oil of the present invention, as seen in Table I, comprises 0.01% to 10% by weight acacia or other tree gum and 90% to 99.99% by weight the cosmetic oil.

TABLE I

| Composition Ingredients | % (by weight) |
|---|---|
| Acacia or another tree gum | 0.01%-10% |
| Existing cosmetic oil | 90%-99.99% |

The viscosity of the fast-absorbing cosmetic oil of the present invention is lowered comparing with the existing cosmetic oil—one of the ingredients used to make the present invention prior to the addition of the tree gum. The preferred range of the viscosity decrease from the existing cosmetic oil to the fast-absorbing cosmetic oil of the present invention is 3 centistokes (cSt) to 30 cSt at a room temperature between 50° F. to 90° F. As can be seen in Table II, an example of the embodiment of the present invention comprises a lower viscosity of 28.6 cSt comparing with the existing cosmetic oil of 34.1 cSt that was used for making the fast-absorbing cosmetic oil of the present invention.

TABLE II

| | Viscosity |
|---|---|
| Example Oils | (centistokes-cSt) |
| An existing cosmetic oil | 34.1 |
| Fast-absorbing cosmetic | 28.6 |
| oil with acacia tree gum | |
| Change of viscosity | 6.5 |

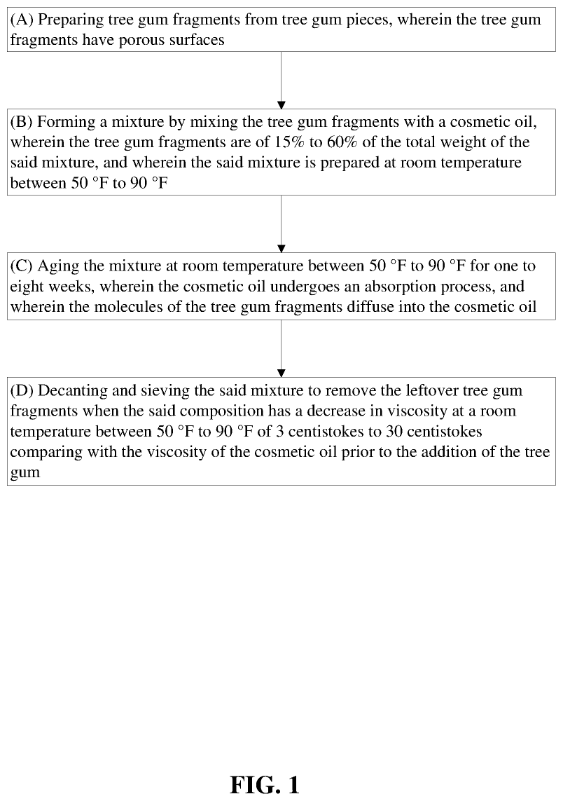

As can be seen in FIG. 1, the preferred embodiment of the present invention provides a method of making the fast-absorbing cosmetic oil without the involvement of water. Thus, this method is also called Dry Method. Specifically, the overall process of the preferred method starts with preparing tree gum fragments from tree gum pieces through sanding or grinding, wherein the tree gum fragments have porous surfaces (Step A). The porous surfaces of tree gum fragments can be prepared by, but not limited to, sanding or grinding the tree gum pieces, or by crushing the tree gum pieces. The tree gum fragments may include, but are not limited to, small pieces, chunks, granules, or powder form. The next step of the overall process is forming a mixture by mixing the tree gum fragments with a cosmetic oil, wherein the tree gum fragments are of 15% to 60% of the total weight of the said mixture, and wherein the said mixture is prepared at room temperature between 50° F. to 90° F. (Step B). Specifically, the right amount of the existing cosmetic oil is poured into the tree gum fragments mixing the oil and the solids. Subsequently, the mixture is left for aging at room temperature between 50° F. to 90° F. for one to eight weeks, wherein the cosmetic oil undergoes an absorption process, and wherein the molecules of the tree gum fragments diffuse into the cosmetic oil (Step C). Finally, the mixture is filtered to obtain the fast-absorbing cosmetic oil by decanting or sieving the said mixture to remove the leftover tree gum fragments when the said composition has a decrease in viscosity at a room temperature between 50° F. to 90° F. of 3 centistokes to 30 centistokes comparing with the viscosity of the cosmetic oil prior to the addition of tree gum (Step D). The measurements of the kinematic viscosities of the existing cosmetic oil used in this process and the fast-absorbing cosmetic oil can be made by an instrument including, but not limited to, capillary tube viscometer, a manual Zahn® cup viscometer, etc.

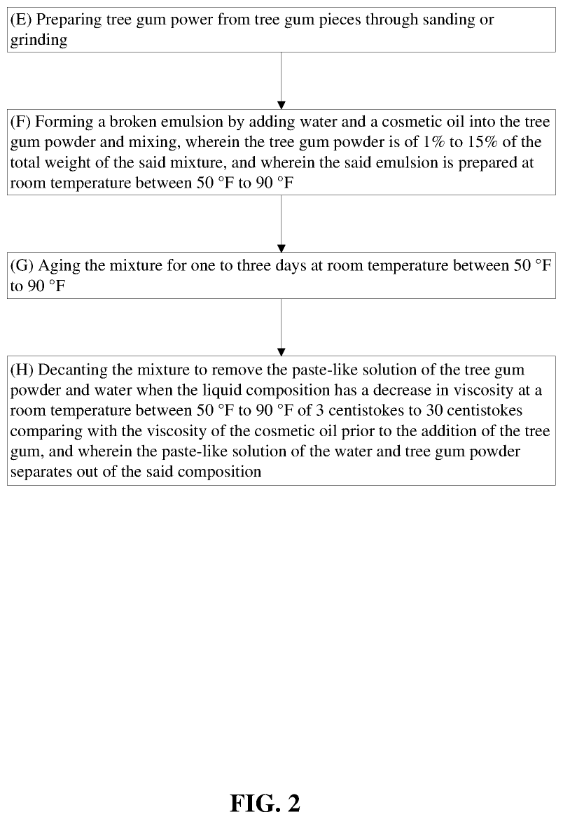

As can be seen in FIG. 2, another embodiment of the present invention provides an alternative method of making the fast-absorbing cosmetic oil through the preparation of a broken emulsion. Thus, this method is called Wet Method. Specifically, the overall process of the alternative method starts with preparing the tree gum powder (Step E), where the tree gum powder can be prepared by, but not limited to, sanding or grinding the tree gum material. The next step is to form a broken emulsion by adding water and a cosmetic oil into the tree gum powder and mixing, wherein the tree gum powder is of 1% to 15% of the total weight of the said mixture, and wherein the said emulsion is prepared at room temperature between 50° F. to 90° F. (Step F). The emulsion is forced to break (or separate), and then the mixture is left for aging for one to three days at room temperature between 50° F. to 90° F. (Step G), until the mixture fully separates, the lipophilic molecules of the tree gum have penetrated the cosmetic oil, and the desired viscosity of the tree gum and cosmetic oil liquid composition is achieved. Finally, the tree gum and cosmetic oil mixture is decanted off to remove the paste-like solution of the combined tree gum powder and water when the liquid composition has a decrease in viscosity at a room temperature between 50° F. to 90° F. of 3 centistokes to 30 centistokes comparing with the viscosity of the cosmetic oil prior to the addition to the tree gum, and wherein the paste-like solution of water and tree gum powder separate out of the said composition (Step H). Consequently, the water molecules bonded to the hydrophilic molecules of the tree gum particles and the cosmetic oil separate out, while the lipophilic molecules of the tree gum particles remain in the cosmetic oil for faster penetrating into and absorption by the user's skin. Furthermore, this alternative method requires less time to make one batch of the present invention than the preferred method of the present invention.

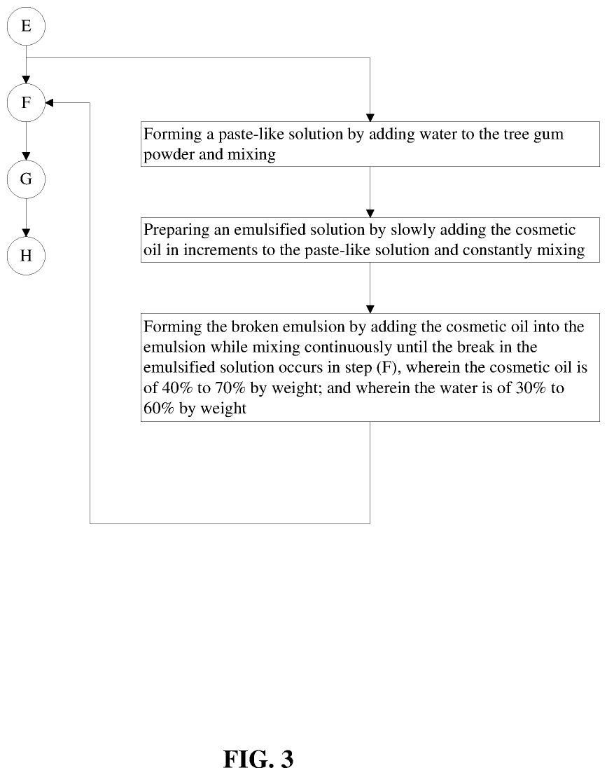

As can be seen in FIG. 3, the alternative method of the present invention provides a preferred sub-process for preparing the broken emulsion in Step F. Specifically, the first step is to form a paste-like solution by adding water to the tree gum powder and mixing. The water and tree gum powder are mixed to form a paste. Then the cosmetic oil is slowly added in small increments while constantly mixing to produce an emulsified solution. The difference in this process from a regular emulsion process is that the said emulsion in this method is purposely forced to break to separate out the solution of the water and the hydrophilic molecules of the tree gum from the solution of the cosmetic oil and the lipophilic molecules of the tree gum. Finally, the broken emulsion is formed by adding the cosmetic oil into the paste-like solution while mixing continuously until the break in the emulsified solution occurs in Step F, wherein the oil is of 40% to 70% by weight; and wherein the water is of 30% to 60% by weight.

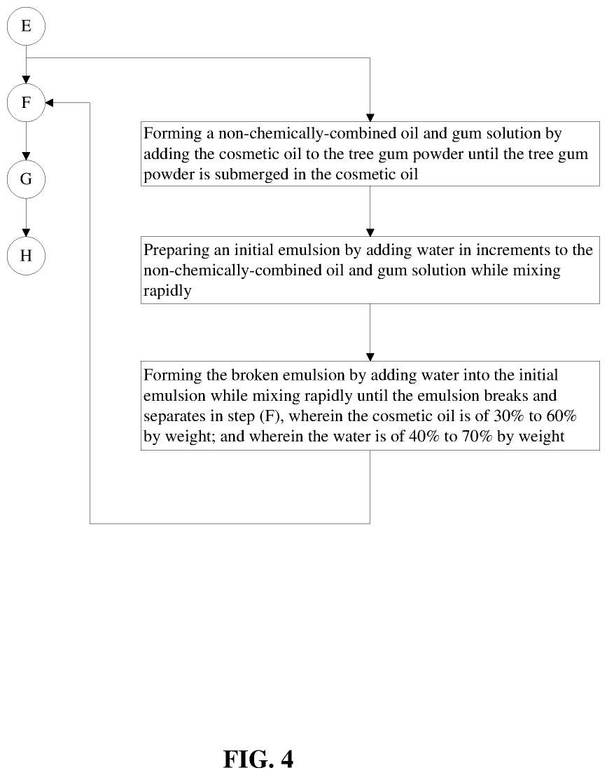

As can be seen in FIG. 4, the alternative method of the present invention provides an alternative sub-process for preparing the broken emulsion in Step F. Specifically, the first step is to form a non-chemically-combined oil and gum solution by adding the cosmetic oil to the tree gum powder until the tree gum powder is submerged in the cosmetic oil. Water is then added in increments into the solution of the cosmetic oil and tree gum powder while rapidly mixing to produce an initial emulsion. Additionally, this initial emulsion is forced to break when more water is added. Finally, the broken emulsion is formed by continuing to add the water until the emulsified solution breaks and the separation begins in Step F, wherein the oil is of 30% to 60% by weight; and wherein the water is of 40% to 70% by weight.

In the preferred and alternative embodiments of the sub-process in Step F, each emulsion is forced to break by adding the second liquid ingredient in a greater amount than the first liquid ingredient until the mixture breaks or separates. Since water solution separates out and is decanted off and the final composition does not comprise water, the preferred sub-process of Step F produces higher quantity of the fast-absorbing cosmetic oil of the present invention in a single batch.

Although the invention has been explained in relation to its preferred embodiment, it is to be understood that many other possible modifications and variations can be made without departing from the spirit and scope of the invention as hereinafter claimed.

What is claimed is:

1. A fast-absorbing cosmetic oil composition comprising:
a tree gum;
a cosmetic oil; and
the tree gum being 0.01% to 10% by weight of the said composition.

2. The fast-absorbing cosmetic oil composition as claimed in claim 1, wherein the said composition has a decrease in viscosity at a room temperature between 50° F. to 90° F. of 3 centistokes to 30 centistokes comparing with the viscosity of the cosmetic oil prior to the addition of the tree gum.

3. The fast-absorbing cosmetic oil composition as claimed in claim 1, wherein the tree gum is an acacia tree gum.

4. The fast-absorbing cosmetic oil composition as claimed in claim 1, wherein the tree gum is a gum ghatti.

5. The fast-absorbing cosmetic oil composition as claimed in claim 1, wherein the tree gum is a gum tragacanth.

6. The fast-absorbing cosmetic oil composition as claimed in claim 1, wherein the tree gum is a karaya gum.

7. A method for preparing a fast-absorbing cosmetic oil composition comprising:
(A) preparing tree gum fragments from tree gum pieces, wherein the tree gum fragments have porous surfaces;
(B) forming a mixture by mixing the tree gum fragments with a cosmetic oil, wherein the tree gum fragments are of 15% to 60% of the total weight of the said mixture, and wherein the said mixture is prepared at room temperature between 50° F. to 90° F.;
(C) aging the mixture at room temperature between 50° F. to 90° F. for one to eight weeks, wherein the cosmetic oil undergoes an absorption process, and wherein the molecules of the tree gum fragments diffuse into the cosmetic oil; and
(D) decanting and sieving the said mixture to remove the leftover tree gum fragments when the said composition has a decrease in viscosity at a room temperature between 50° F. to 90° F. of 3 centistokes to 30 centistokes comparing with the viscosity of the cosmetic oil prior to the addition of the tree gum.

8. The method for preparing a fast-absorbing cosmetic oil composition as claimed in claim 7, wherein the tree gum is prepared in powder form in step (A).

9. A method for preparing a fast-absorbing cosmetic oil composition as claimed in claim 7 comprising:
(E) preparing tree gum power from tree gum pieces through sanding or grinding;
(F) forming a broken emulsion by adding water and a cosmetic oil into the tree gum powder and mixing, wherein the tree gum powder is of 1% to 15% of the total weight of the said mixture, and wherein the said emulsion is prepared at room temperature between 50° F. to 90° F.;
(G) aging the mixture for one to three days at room temperature between 50° F. to 90° F.; and
(H) decanting the mixture to remove the paste-like solution of the tree gum powder and water when the liquid composition has a decrease in viscosity at a room temperature between 50° F. to 90° F. of 3 centistokes to 30 centistokes comparing with the viscosity of the cosmetic oil prior to the addition of the tree gum, and wherein the paste-like solution of the water and tree gum powder separates out of the said composition.

10. The method for preparing a fast-absorbing cosmetic oil composition as claimed in claim 9 comprising:
forming a paste-like solution by adding water to the tree gum powder and mixing;
preparing an emulsified solution by slowly adding the cosmetic oil in increments to the paste-like solution and constantly mixing; and
forming the broken emulsion by adding the cosmetic oil into the emulsion while mixing continuously until the break in the emulsified solution occurs in step (F), wherein the cosmetic oil is of 40% to 70% by weight; and wherein the water is of 30% to 60% by weight.

11. The method for preparing a fast-absorbing cosmetic oil composition as claimed in claim 9 comprising:
forming a non-chemically-combined oil and gum solution by adding the cosmetic oil to the tree gum powder until the tree gum powder is submerged in the cosmetic oil;
preparing an initial emulsion by adding water in increments to the non-chemically-combined oil and gum solution while mixing rapidly; and
forming the broken emulsion by adding water into the initial emulsion while mixing rapidly until the emulsion breaks and separates in step (F), wherein the cosmetic oil is of 30% to 60% by weight; and wherein the water is of 40% to 70% by weight.
